(19) United States
(12) Patent Application Publication
(10) Pub. No.: US 2023/0181910 A1
(43) Pub. Date: Jun. 15, 2023

(54) PACING AND SENSING DEVICES AND CONTROL SYSTEM

(71) Applicant: MAXWELL BIOMEDICAL, INC., San Diego, CA (US)

(72) Inventor: RANDELL L. WERNETH, San Diego, CA (US)

(21) Appl. No.: 17/925,821

(22) PCT Filed: Jun. 1, 2021
(86) PCT No.: PCT/US2021/035132

§ 371 Date: Nov. 16, 2022

Related U.S. Application Data
(60) Provisional application No. 63/032,687, filed on May 31, 2020.

(51) Int. Cl. A61N 1/362; A61N 1/372

(52) U.S. Cl. CPC A61N 1/3621; A61N 1/37247

(57) ABSTRACT

Systems for treating a cardiac condition of a patient are provided. The system comprises an implantable device for delivering energy to the patient’s heart, and an external patient device configured to wirelessly communicate with the implantable device. The system can further comprise a clinician device for implanting the implantable device in the patient. The cardiac condition treated by the system can comprise atrial fibrillation. Methods of treating a cardiac condition are also provided.

### RELATED APPLICATIONS

The present application claims priority to U.S. Provisional Pat. Application Serial No. 63/032,687, entitled “Rechargeable Biomedical Battery Powered Wireless Self-Anchoring Micro-pacing and Sensing Devices and Control System”, filed May 31, 2020, the content of which is incorporated herein by reference in its entirety for all purposes.

The present application, while not claiming priority to, may be related to International PCT Patent Application Serial Number PCT/US2020/049349, entitled “Cardiac Stimulation System”, filed Sep. 4, 2020, Publication Number WO 2021/046313, published Mar. 11, 2021, which claims priority to U.S. Provisional Application Serial No. 62/895,655, filed Sep. 4, 2019, each of which is hereby incorporated by reference.

The present application, while not claiming priority to, may be related to International PCT Patent Application Serial Number PCT/US2021/021467, entitled “Cardiac Pacing Device”, filed Mar. 9, 2021, Publication Number ______, published ______, which claims priority to U.S. Provisional Pat. Application Serial No. 62/987,238, entitled “Stent, Mounted and Delivered Wireless, Batteryless Micropacing Chip System”, filed Mar. 9, 2020, each of which is hereby incorporated by reference.

### TECHNICAL FIELD

The present inventive concepts relate generally to medical systems, and in particular systems and devices used to pace or otherwise treat the heart of a patient.

### BACKGROUND

The heart is a critical muscle in humans and many other animals that is responsible for circulating blood through the circulatory system. The human heart is made up of four chambers, two upper atria, and two lower ventricles, organized into a left and right pairing of an atrium and an associated ventricle. In a healthy heart, the chambers contract and relax in a synchronized fashion, referred to as a “beat,” in order to force blood through the network of veins and arteries.

Irregular heartbeats can pose a health risk, and in some cases regular beating can be restored via electrical stimulation. Implantable devices called “pacemakers” are devices which can stimulate the muscle tissue, causing it to contract. By carefully and regularly applying stimulation as needed, normal heart rhythm can be restored.

There is a need for improved systems for treating irregular heartbeats.

### BRIEF SUMMARY

According to an aspect of the present inventive concepts, a system for treating a cardiac condition of a patient comprises an implantable device for delivering energy to the patient’s heart, an external patient device configured to wirelessly communicate with the implantable device, and a clinician device for implanting the implantable device in the patient. The cardiac condition treated by the system can comprise atrial fibrillation.

In some embodiments, the system is configured to provide electrical energy to the heart to perform a therapeutic and/or a diagnostic procedure on the patient.

In some embodiments, the system is further configured to map electrical activity of the patient’s heart.

In some embodiments, the system is further configured to deliver ablative energy to heart tissue of the patient.

In some embodiments, the implantable device is configured to deliver the energy while varying pulse rate, duration, and/or amplitude of electrical pulses of the energy delivered.

In some embodiments, the energy delivered by the implantable device comprises low voltage rhythmic electrical signals.

In some embodiments, at least a portion of the implantable device is configured to be implanted in the vein of Marshall.

In some embodiments, at least a portion of the implantable device is configured to be implanted on an epicardial surface of the patient’s heart.

In some embodiments, at least a portion of the implantable device is configured to be implanted in an anatomical location selected from the group consisting of: a coronary vein; a coronary artery; the vein of Marshall; the coronary sinus; the great cardiac vein; the anterior intraventricular vein; the middle cardiac vein; and combinations thereof.

In some embodiments, the implantable device comprises a first portion configured to be implanted in a first anatomic location and a second portion configured to be implanted in a second anatomic location. The first anatomic location can comprise the vein of Marshall. The first anatomic location can comprise an epicardial surface of the heart. The first anatomic location can comprise the coronary sinus. The second anatomic location can comprise the vein of Marshall. The second anatomic location can comprise an epicardial surface of the heart.

In some embodiments, the implantable device is configured to deliver the energy to multiple sites on the heart. The implantable device can be configured to deliver the energy to the multiple sites using synchronous delivery of energy, asynchronous delivery of energy, or both synchronous and asynchronous delivery of energy. The delivery of energy can comprise multi-site burst pacing delivery of energy. The energy delivery can comprise pacing rates that can be set in proportion to AF cycle lengths.

In some embodiments, the implantable device comprises a power supply. The power supply can comprise a lithium ion polymer battery.

In some embodiments, the implantable device comprises a substrate comprising one or more substrates. The substrate can comprise one or more flexible PCBs. The substrate can comprise a coating. The coating can comprise a polymeric coating. The substrate can comprise a dimension selected from the group consisting of: a length of 22 mm; a width of 1.5 mm; a thickness of 0.4 mm; and combinations thereof. The substrates can comprise a PCB including 36 µm copper thickness. The substrate can comprise a multi-layer PCB. The multi-layer PCB can comprise an antenna. The substrate can comprise an electrode array comprising one or more electrodes. The electrode array can comprise at least one electrode with a curved geometry. The electrode array can comprise at least one electrode comprising a flexible electrode. The electrode array can be configured to transition from a collapsed geometry to an expanded geometry. The implantable device can comprise one or more anchoring elements each comprising a projection that extends from the substrate.

In some embodiments, the implantable device comprises an anchor comprising one or more anchoring elements. The anchor can comprise a stent-like component. The anchor can comprise a self-expanding and/or a balloon-expandable component. The anchor can comprise a first end portion, a second end portion, and a middle portion therebetween, and the first end portion and the second end portion can be configured to radially expand. The middle portion can be configured to axially extend. The anchor can comprise a ribbon coil geometry. The anchor can comprise a loop geometry.

In some embodiments, the implantable device comprises an antenna assembly comprising one or more antennas. The antenna assembly can comprise a planar micro coil antenna.

In some embodiments, the implantable device comprises a power module configured to harvest power. The power module can be configured to harvest power provided by a separate component of the system. The power module can comprise a piezoelectric component configured to convert kinetic energy to electrical energy.

In some embodiments, the implantable device comprises a sensor comprising one or more sensors. The sensor can comprise one, two, or more sensors selected from the group consisting of: accelerometer; position sensor; gravimetric sensor; pressure sensor; strain gauge; and combinations thereof. The sensor can comprise one, two, or more sensors selected from the group consisting of: pressure sensor; blood pressure sensor; acoustic sensor; respiration sensor; gas sensor; blood gas sensor; flow sensor; blood flow sensor; temperature sensor; pH sensor; optical sensor; metabolic sensor; gravitational position sensor, gravitational orientation sensor, physical motion sensor; body position sensor; and combinations thereof.

In some embodiments, the external patient device is configured to wirelessly transmit data, power, or both data and power to the implantable device. The external patient device can be configured to be worn by the patient. The external patient device can comprise an antenna.

In some embodiments, the system further comprises a console configured to operably connect to the clinician device. The console can comprise a mapping module, an energy delivery module, or both a mapping module and an energy delivery module.

In some embodiments, the system further comprises an algorithm configured to analyze data produced by the system. The algorithm can comprise an artificial intelligence algorithm. The algorithm can be configured to analyze data from multiple patients.

In some embodiments, the implantable device comprises an electrode array comprising one or more electrodes configured to deliver the energy to the patient’s heart. The one or more electrodes can comprise one or more microneedles.

### INCORPORATION BY REFERENCE

All publications, patents, and patent applications mentioned in this specification are herein incorporated by reference to the same extent as if each individual publication, patent, or patent application was specifically and individually indicated to be incorporated by reference. The content of all publications, patents, and patent applications mentioned in this specification are herein incorporated by reference in their entirety for all purposes.

### BRIEF DESCRIPTION OF THE DRAWINGS

FIG. 1 illustrates a schematic view of a system for diagnosing and/or treating a patient, consistent with the present inventive concepts.

FIG. 2 illustrates a graphic of an action potential illustrating an atrial fibrillation cycle length, consistent with the present inventive concepts.

FIG. 3 illustrates a photographic view of an array of electrodes, consistent with the present inventive concepts.

FIG. 4 illustrates a method for diagnosing and treating atrial fibrillation, consistent with the present inventive concepts.

FIG. 5 illustrates a method for implanting a stent anchor, consistent with the present inventive concepts.

FIGS. 6A and 6B illustrate side views of an assembly process for an implantable device, consistent with the present inventive concepts.

FIG. 7 illustrates a perspective view of an anchor, consistent with the present inventive concepts.

FIGS. 8A and 8B illustrate top and side anatomical views of an implantable device, respectively, consistent with the present inventive concepts.

FIG. 9 illustrates an anchor comprising a ribbon coil, consistent with the present inventive concepts.

FIG. 10 illustrates a top view of an implantable device, consistent with the present inventive concepts.

FIG. 11 illustrates a photographic view of an implantable device and a delivery catheter, consistent with the present inventive concepts.

FIGS. 12A and 12B illustrate side views of an assembly process for an implantable device, consistent with the present inventive concepts.

FIG. 13 illustrates a perspective view of an implantable device including ring-shaped anchors, consistent with the present inventive concepts.

FIG. 14 illustrates a perspective view of an implantable device including curved anchors, consistent with the present inventive concepts.

FIGS. 15A-B illustrate a top view of an implantable device, and a perspective view of a portion of the implantable device, respectively, consistent with the present inventive concepts.

### DETAILED DESCRIPTION OF THE DRAWINGS

Reference will now be made in detail to the present embodiments of the technology, examples of which are illustrated in the accompanying drawings. Similar reference numbers may be used to refer to similar components. However, the description is not intended to limit the present disclosure to particular embodiments, and it should be construed as including various modifications, equivalents, and/or alternatives of the embodiments described herein.

It will be understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be further understood that, although the terms first, second, third, etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g. “between” versus “directly between,” “adjacent” versus “directly adjacent,” etc.).

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of one or more of these.

As used herein, the term “proximate”, when used to describe proximity of a first component or location to a second component or location, is to be taken to include one or more locations near to the second component or location, as well as locations in, on and/or within the second component or location. For example, a component positioned proximate an anatomical site (e.g. a target tissue location), shall include components positioned near to the anatomical site, as well as components positioned in, on and/or within the anatomical site.

Spatially relative terms, such as “beneath,” “below,” “lower,” “above,” “upper” and the like may be used to describe an element and/or feature’s relationship to another element(s) and/or feature(s) as, for example, illustrated in the figures. It will be further understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g. rotated 90 degrees or at other orientations) and the spatially relative descriptors used herein interpreted accordingly.

The terms “reduce”, “reducing”, “reduction” and the like, where used herein, are to include a reduction in a quantity, including a reduction to zero. Reducing the likelihood of an occurrence shall include prevention of the occurrence. Correspondingly, the terms “prevent”, “preventing”, and “prevention” shall include the acts of “reduce”, “reducing”, and “reduction”, respectively.

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

The term “one or more”, where used herein can mean one, two, three, four, five, six, seven, eight, nine, ten, or more, up to any number.

The terms “and combinations thereof” and “and combinations of these” can each be used herein after a list of items that are to be included singly or collectively. For example, a component, process, and/or other item selected from the group consisting of: A; B; C; and combinations thereof, shall include a set of one or more components that comprise: one, two, three or more of item A; one, two, three or more of item B; and/or one, two, three, or more of item C.

In this specification, unless explicitly stated otherwise, “and” can mean “or”, and “or” can mean “and”. For example, if a feature is described as having A, B, or C, the feature can have A, B, and C, or any combination of A, B, and C. Similarly, if a feature is described as having A, B, and C, the feature can have only one or two of A, B, or C.

As used herein, when a quantifiable parameter is described as having a value “between” a first value X and a second value Y, it shall include the parameter having a value of: at least X, no more than Y, and/or at least X and no more than Y. For example, a length of between 1 and 10 shall include a length of at least 1 (including values greater than 10), a length of less than 10 (including values less than 1), and/or values greater than 1 and less than 10.

The expression “configured (or set) to” used in the present disclosure may be used interchangeably with, for example, the expressions “suitable for”, “having the capacity to”, “designed to”, “adapted to”, “made to” and “capable of” according to a situation. The expression “configured (or set) to” does not mean only “specifically designed to” in hardware. Alternatively, in some situations, the expression “a device configured to” may mean that the device “can” operate together with another device or component.

As used herein, the term “threshold” refers to a maximum level, a minimum level, and/or range of values correlating to a desired or undesired state. In some embodiments, a system parameter is maintained above a minimum threshold, below a maximum threshold, within a threshold range of values, and/or outside a threshold range of values, such as to cause a desired effect (e.g. efficacious therapy) and/or to prevent or otherwise reduce (hereinafter “prevent”) an undesired event (e.g. a device and/or clinical adverse event). In some embodiments, a system parameter is maintained above a first threshold (e.g. above a first temperature threshold to cause a desired therapeutic effect to tissue) and below a second threshold (e.g. below a second temperature threshold to prevent undesired tissue damage). In some embodiments, a threshold value is determined to include a safety margin, such as to account for patient variability, system variability, tolerances, and the like. As used herein, “exceeding a threshold” relates to a parameter going above a maximum threshold, below a minimum threshold, within a range of threshold values and/or outside of a range of threshold values.

As described herein, “room pressure” shall mean pressure of the environment surrounding the systems and devices of the present inventive concepts. Positive pressure includes pressure above room pressure or simply a pressure that is greater than another pressure, such as a positive differential pressure across a fluid pathway component such as a valve. Negative pressure includes pressure below room pressure or a pressure that is less than another pressure, such as a negative differential pressure across a fluid component pathway such as a valve. Negative pressure can include a vacuum but does not imply a pressure below a vacuum. As used herein, the term “vacuum” can be used to refer to a full or partial vacuum, or any negative pressure as described hereabove.

The term “diameter” where used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross sectional area as the cross section of the component being described.

The terms “major axis” and “minor axis” of a component where used herein are the length and diameter, respectively, of the smallest volume hypothetical cylinder which can completely surround the component.

As used herein, the term “functional element” is to be taken to include one or more elements constructed and arranged to perform a function. A functional element can comprise a sensor and/or a transducer. In some embodiments, a functional element is configured to deliver energy and/or otherwise treat tissue (e.g. a functional element configured as a treatment element). Alternatively or additionally, a functional element (e.g. a functional element comprising a sensor) can be configured to record one or more parameters, such as a patient physiologic parameter; a patient anatomical parameter (e.g. a tissue geometry parameter); a patient environment parameter; and/or a system parameter. In some embodiments, a sensor or other functional element is configured to perform a diagnostic function (e.g. to gather data used to perform a diagnosis). In some embodiments, a functional element is configured to perform a therapeutic function (e.g. to deliver therapeutic energy and/or a therapeutic agent). In some embodiments, a functional element comprises one or more elements constructed and arranged to perform a function selected from the group consisting of: deliver energy; extract energy (e.g. to cool a component); deliver a drug or other agent; manipulate a system component or patient tissue; record or otherwise sense a parameter such as a patient physiologic parameter or a system parameter; and combinations of one or more of these. A functional element can comprise a fluid and/or a fluid delivery system. A functional element can comprise a reservoir, such as an expandable balloon or other fluid-maintaining reservoir. A “functional assembly” can comprise an assembly constructed and arranged to perform a function, such as a diagnostic and/or therapeutic function. A functional assembly can comprise an expandable assembly. A functional assembly can comprise one or more functional elements.

The term “transducer” where used herein is to be taken to include any component or combination of components that receives energy or any input, and produces an output. For example, a transducer can include an electrode that receives electrical energy, and distributes the electrical energy to tissue (e.g. based on the size of the electrode). In some configurations, a transducer converts an electrical signal into any output, such as: light (e.g. a transducer comprising a light emitting diode or light bulb), sound (e.g. a transducer comprising a piezo crystal configured to deliver ultrasound energy); pressure (e.g. an applied pressure or force); heat energy; cryogenic energy; chemical energy; mechanical energy (e.g. a transducer comprising a motor or a solenoid); magnetic energy; and/or a different electrical signal (e.g. different than the input signal to the transducer). Alternatively or additionally, a transducer can convert a physical quantity (e.g. variations in a physical quantity) into an electrical signal. A transducer can include any component that delivers energy and/or an agent to tissue, such as a transducer configured to deliver one or more of: electrical energy to tissue (e.g. a transducer comprising one or more electrodes); light energy to tissue (e.g. a transducer comprising a laser, light emitting diode and/or optical component such as a lens or prism); mechanical energy to tissue (e.g. a transducer comprising a tissue manipulating element); sound energy to tissue (e.g. a transducer comprising a piezo crystal); chemical energy; electromagnetic energy; magnetic energy; and combinations of one or more of these.

As used herein, the term “fluid” can refer to a liquid, gas, gel, or any flowable material, such as a material which can be propelled through a lumen and/or opening.

As used herein, the term “material” can refer to a single material, or a combination of two, three, four, or more materials.

It is appreciated that certain features of the inventive concepts, which are, for clarity, described in the context of separate embodiments, may also be provided in combination in a single embodiment. Conversely, various features of the inventive concepts which are, for brevity, described in the context of a single embodiment, may also be provided separately or in any suitable sub-combination. For example, it will be appreciated that all features set out in any of the claims (whether independent or dependent) can be combined in any given way.

It is to be understood that at least some of the figures and descriptions of the inventive concepts have been simplified to focus on elements that are relevant for a clear understanding of the inventive concepts, while eliminating, for purposes of clarity, other elements that those of ordinary skill in the art will appreciate may also comprise a portion of the inventive concepts. However, because such elements are well known in the art, and because they do not necessarily facilitate a better understanding of the inventive concepts, a description of such elements is not provided herein.

Terms defined in the present disclosure are only used for describing specific embodiments of the present disclosure and are not intended to limit the scope of the present disclosure. Terms provided in singular forms are intended to include plural forms as well, unless the context clearly indicates otherwise. All of the terms used herein, including technical or scientific terms, have the same meanings as those generally understood by an ordinary person skilled in the related art, unless otherwise defined herein. Terms defined in a generally used dictionary should be interpreted as having meanings that are the same as or similar to the contextual meanings of the relevant technology and should not be interpreted as having ideal or exaggerated meanings, unless expressly so defined herein. In some cases, terms defined in the present disclosure should not be interpreted to exclude the embodiments of the present disclosure.

The present inventive concepts generally relate to implantable cardiac therapy systems, devices, and methods, and, more particularly, to wireless cardiac therapies involving controlled delivery of electrical stimulation energy to a patient’s heart, such as for treatment of arrhythmias and/or for pacing of the heart via a system for delivering such therapies.

The system of the present inventive concepts can be configured as a pacemaker, a cardioverter-defibrillator, or both. The system can provide electrical energy (e.g. pulses) to the heart, such as to perform a therapeutic and/or diagnostic function.

For diagnostics, one or more electrical pulses can be delivered to the heart, and measured effects from the one or more pulses provide diagnostic feedback.

For therapeutics, one or more electrical pulses can be delivered to the heart, via a synchronous and/or asynchronous delivery. The electrical pulses can be delivered while varying the pulse rate, duration and/or amplitude. The therapeutic effect can be to restore normal sinus rhythm.

Depending upon the various events sensed by the system, the system can stimulate the left atrium, the left ventricle, and/or both left chambers of the heart (e.g. in succession).

In the treatment of a chronic cardiac condition, such as atrial arrhythmias, a challenge posed is that the patient typically is conscious during the arrhythmia and can potentially perceive any programmed electrical stimulation treatment being performed on their heart. For example, one current method of electrical shock therapy for treating atrial (or ventricular) arrhythmia is to deliver a single burst of a relatively large amount of electrical current through the fibrillating heart of a patient. For a given atrial fibrillation (AF) episode, the minimum amount of energy required to defibrillate a patient’s atrium is known as the atrial defibrillation/pacing threshold (ADFT).

Pacing systems of the present inventive concepts can produce and deliver low voltage rhythmic electrical signals that remedy a diseased heart’s defective ability to generate its own electrical signals, or to re-orientate the aberrant electrical signals (e.g. aberrant signals which may cause the heart to beat too fast, too slow, and/or irregularly). The pacing systems described herein can continuously monitor the heart’s electrical system, and deliver one or more electrical pulses to aid the heart when the system detects a need for it. Today’s cardiac pacing systems are used to treat bradyarrhythmia or bradycardia, which is when the heart beats too slowly due to a defect in the sinoatrial node or a blockage in the heart’s own electrical conduction system, thus reducing blood flow and prohibiting the body from receiving the blood it needs. Atrial Fibrillation is an arrhythmia in which a system of the present inventive concepts would be a significant benefit to patients (e.g. when the system delivers treatment at a level below one Joule of energy).

The system of the present inventive concepts can include one or more components (e.g. at least a portion of one or more components) that are positioned in the vein of Marshall (a vein on the outside of the left atrium).

The system of the present inventive concepts can be configured to be implanted in a patient in a procedure that can be performed in approximately 30 to 45 minutes.

The system of the present inventive concepts can include a catheter that can be inserted into the inferior vena cava (IVC), and then sequentially advanced into the right atrium, the coronary sinus, and into the vein of Marshall (VoM). Once positioned in the VoM, a flexible printed circuit board (a “PCB”, such as a PCB including multiple pacing electrodes and electronic circuits) can be delivered from the catheter. Once released from the catheter, the PCB expands into the VoM. Before being released from the catheter, a diagnostic procedure can be performed, such as to establish a patient baseline that can be used in phenotyping of the atrial fibrillation, and/or used to perform an optimization of a therapeutic pacing algorithm.

Regular patient checkups can be performed to assess the life of the battery (e.g. to determine when a re-charging should be performed), and/or to update any system programming as desired.

The present inventive concepts provide numerous advantages as compared to current commercially available devices. An implanted device of the system described herein can include a power supply, such as a battery made of lithium ion polymer (LiPo), or similar, such as to provide energy (e.g. via a wired or wireless connection) to implanted “micropacing chips” (also referred to as “micropacing chip assembly” herein) that can comprise electronic circuitry and electrodes for delivering energy to tissue. The system of the present inventive concepts can be configured to perform various sensing functions, and when needed, transmit energy (e.g. wirelessly) to one or more micropacing chips for therapy delivery.

The systems of the present inventive concepts can include a power supply comprising a battery, capacitor, and/or other energy storage device that is capable of being recharged, such as to maintain life-long, relatively (e.g. substantially) continuous use.

The systems of the present inventive concepts can deliver an amount of energy that is therapeutically effective, but at a level low enough to improve battery life, and/or be below a pain threshold for the patient. The micropacing chip assembly can require an amount of energy less than a traditional implantable cardiac defibrillator (ICD), such as to provide a reduced power requirement that is less than half that of a current ICD.

As described hereabove, each micropacing chip assembly can be constructed on a flexible PCB, and it can be delivered into the vein of Marshall (VoM). A wireless energy harvesting circuit (e.g. a circuit configured to received radiofrequency, ultrasound, and/or other energy from a separate device) can be positioned in the coronary sinus. The flexible PCB can be encapsulated within a coating that hermetically seals the components of the PCB assembly (e.g. to protect those components from the surrounding biologic environment). For example, the micropacing device can comprise a polymeric coating that electrically insulates and seals the electronic and/or other componentry of the implanted assemblies, such as a parylene and/or other polymeric coating. The micropacing chip assembly can comprise a stent-like component and/or other component configured to anchor the assembly in the patient (e.g. to prevent migration). In some embodiments, the system of the present inventive concepts, and/or one or more of its components, are of similar construction and/arrangement to those described in “Systems and Methods for Controlling Wirelessly Powered Leadless Pacemakers” PCT R4-06361 filed by UCLA (International PCT Patent Application Serial No. PCT/US2019/062443 entitled “Systems and Methods for Controlling Wirelessly Powered Leadless Pacemakers” filed Nov. 20, 2019, Provisional Pat. Application No. 62/769,984 entitled “Synchronized Biventricular Heart Pacing using Wirelessly powered, leadless pacemakers” filed Nov. 20, 2018, and U.S. Provisional Pat. Application No. 62/845,619 entitled “Synchronized Biventricular Heart Pacing using Wirelessly powered, leadless pacemakers” filed May 9, 2019. The disclosures of International PCT Patent Application No. 62/845,619, as well as U.S. Provisional Pat. Application Nos. 62/769,984 and 62/845,619, are hereby incorporated by reference in its entirety for all purposes).

The present inventive concepts provide methods, systems, and devices for achieving normal sinus rhythm in patients with atrial fibrillation and/or another arrhythmia. Atrial pacing can be first conducted from multiple pacing sites in a synchronous and/or asynchronous manner, such as to have the desired effect of maximizing the resynchronizing of atrial tissue. Next, multi-site burst pacing can be introduced, such as to achieve atrial rhythm correction. Burst pacing energy requirements have been shown to be dramatically reduced when using multiple burst pacing sites and pacing rates set proportionally to the sensed atrial fibrillation cycle length(s), such that large areas of atrial tissues are phase-locked, and consequently atrial defibrillation can be effected in the patient with greatly reduced energy requirements.

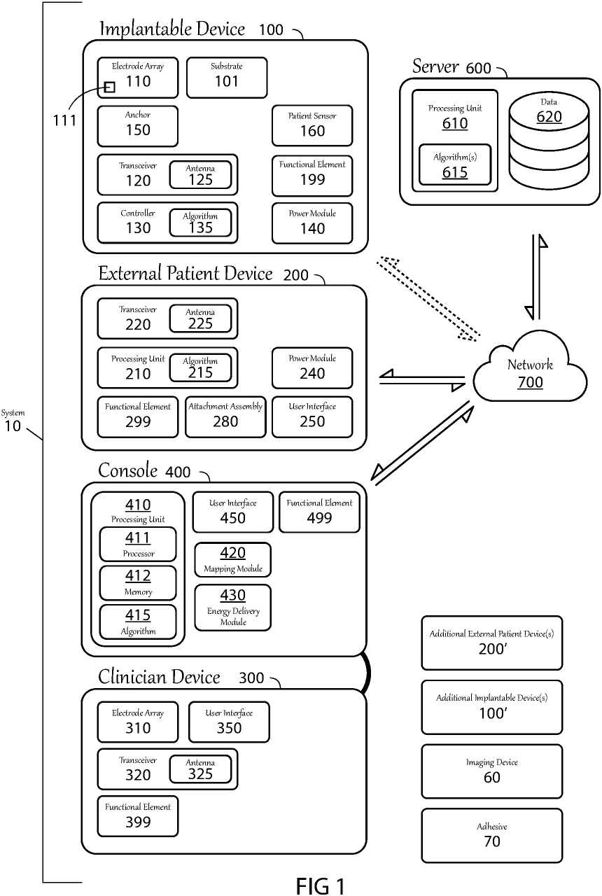

Referring now to FIG. 1, a schematic view of a system for diagnosing and/or treating a patient is illustrated, consistent with the present inventive concepts. System 10 can comprise one or more devices (e.g. devices for a clinician to perform a procedure, devices for a patient to position proximate their body, and/or devices for implantation in the patient) which can be configured to: monitor one or more patient parameters, diagnose one or more patient conditions, and/or to treat one or more patient conditions, such as to treat a condition based on one or more patient diagnoses determined by system 10. For example, system 10 can be configured to diagnose and/or treat (singly, or collectively “treat” or “diagnose” herein) atrial fibrillation (AF), such as by monitoring the electrical activity of the patient’s heart, and by pacing the heart muscle to restore sinus rhythm when fibrillation is detected. System 10 can include one or more devices configured to be implanted, implantable device 100 shown, which can be implanted into the patient for an extended period of time (e.g. at least 1 month, at least 3 months, and/or at least 6 months), such as when implanted by a clinician during a clinical procedure. In some embodiments, system 10 comprises one or more externally-placed devices, external patient device 200 shown, which can comprise one or more devices that are configured to diagnose and/or treat a patient from one or more locations outside the patient’s body. Alternatively or additionally, external patient device 200 (also referred to as EPD 200) can be configured to communicate (e.g. wirelessly communicate) with implantable device 100 (also referred to as ID 100), such as to transfer data between EPD 200 and ID 100, and/or to transfer power from EPD 200 to ID 100.

System 10 can be configured to detect irregular and/or otherwise undesirable (“irregular” or “undesirable” herein) electrical conduction patterns in tissue (e.g. heart tissue) and/or to deliver energy to tissue to restore a regular (e.g. healthy) electrical conduction pattern. For example, system 10 can be configured to monitor the electrical activity of the heart (e.g. conduction patterns proximate the left atrium of the heart), and to detect the presence of irregular and/or otherwise undesired conduction patters, such as conduction patterns that are indicative of AF. Additionally or alternatively, system 10 can deliver electrical energy (e.g. pacing pulses) to tissue to alter the irregular conduction patterns within the tissue. In some embodiments, system 10 is configured to deliver “multi-site” pacing, where pacing energy is delivered from two or more electrodes positioned at different anatomical locations, such as different locations that are each proximate the left atrium. For example, system 10 can be configured to deliver multi-site left-atrial pacing (i.e. delivery of energy to two different left atrial tissue locations) that is configured to restore sinus rhythm in patients experiencing AF.

In some embodiments, system 10 comprises one or more devices (e.g. devices 100 and/or 300 described herein) that are configured to ablate tissue, such as by delivering energy configured to thermally ablate and/or irreversibly electroporate tissue (e.g. tissue associated with atrial fibrillation or other arrhythmia of the patient). In these embodiments, system 10 can be further configured to also deliver pacing energy to tissue, such as multi-site pacing energy and/or other pacing energy, such as is described herein.

System 10 can include one or more devices for use by a clinician during a clinical procedure, clinician device 300. Clinician device 300 (also referred to as CD 300) can comprise one or more delivery devices, such as a kit of devices configured to enable the clinician to perform an implantation procedure in which ID 100 is implanted into the patient. For example, CD 300 can comprise one or more delivery catheters, such as when ID 100 is configured to be implanted during a minimally invasive procedure, such as an interventional procedure performed in a catheterization laboratory (also referred to as a “cath lab”). CD 300 can comprise one, two, or more guidewires, such as to be used in an over-the-wire delivery of a system 10 component. For example, CD 300 can comprise one or more tools for percutaneous delivery of ID 100 in the patient’s vasculature. Alternatively or additionally, CD 300 can comprise one or more surgical tools (e.g. minimally invasive tools) for surgically implanting ID 100 (e.g. in an operating room). In some embodiments, ID 100 comprises a first geometry where ID 100 is in an undeployed state, such as a geometry comprising a collapsed or otherwise compacted geometry configured to allow ease of insertion into the patient. ID 100 can be configured to transition from the first geometry into a second geometry in which ID 100 is in an expanded or otherwise deployed state.

System 10 can include console 400. Console 400 can operably connect to CD 300 and can be configured to facilitate one or more processes, energy deliveries, data collections, data analyses, data transfers, signal processing, and/or other functions (“functions” herein) of system 10. In some embodiments, system 10 is constructed and arranged to map electrical activity within the body (e.g. electrical activity of the heart), such as when CD 300 comprises a mapping catheter and console 400 comprises mapping module 420. Mapping module 420 can be configured to record and/or process mapping signals recorded by CD 300. In some embodiments, system 10 is constructed and arranged to ablate tissue (e.g. ablate cardiac tissue to treat AF). In these embodiments, console 400 comprises energy delivery module 430. Energy delivery module 430 can be configured to deliver ablative energy to tissue, such as via one or more energy delivery elements (e.g. electrodes, ultrasound transducers, light-emitting elements, and the like) of CD 300. Console 400 can include processing unit 410, which can be configured to perform one or more functions of console 400 (e.g. as described hereabove). Processing unit 410 can include processor 411, memory 412, and/or algorithm 415, each as shown. In some embodiments, console 400 includes one or more user interfaces, user interface 450. In some embodiments, console 400 includes one or more functional elements, functional element 499 shown. Functional element 499 can include one or more sensors and/or transducers. Functional element 499 can comprise a pumping mechanism, such as a mechanism configured to deliver a pharmaceutical drug, a cooling or warming fluid, an insufflation fluid, and/or other flowable material (e.g. to clinician device 300).

System 10 can include one or more imaging devices, imaging device 60. Imaging device 60 can comprise an imaging device selected from the group consisting of: an X-ray device such as a fluoroscopy device; a CT scanner device; an MRI device; an ultrasound imaging device; and combinations of these.

Implantable device 100 can comprise one or more substrates, substrate 101 shown. Substrate 101 can comprise one, two, or more printed circuit boards (PCBs). Substrate 101 can be flexible (e.g. a flexible PCB), and/or include one or more flexible and/or hinged portions. Various components of ID 100 can be operably connected to substrate 101 (e.g. and electrically connected to each other via conductive traces of substrate 101). In some embodiments, substrate 101 comprises one, two, or more dimensions selected from the group consisting of: a length of 22 mm; a width of 1.5 mm, and/or a thickness of 0.4 mm. Substrate 101 can comprise a PCB including 36 µm copper thickness. In some embodiments, substrate 101 comprises a multi-layer PCB, such as when multiple layers (e.g. four layers) are used to create a coil, antenna, and/or other component, such as are described herein.

ID 100 can comprise one or more arrays of functional elements (e.g. sensors and/or transducers), electrode array 110, comprising one, two or more elements, electrodes 111. In some embodiments, one or more components of ID 100 (e.g. the components on the outer surfaces of ID 100 which will be exposed to the environment within the body when implanted) comprise biocompatible materials. Electrodes 111 can comprise a flat and/or curved geometry, and each electrode 111 can have square and/or round corners. In some embodiments, one or more electrodes 111 are flexible, such as to be introduced into the patient in a first geometry (e.g. a collapsed geometry), and deployed and implanted in a second geometry (e.g. an expanded geometry). In some embodiments, electrode array 110 is flexible and/or comprises one or more flexible and/or hinged portions, such that electrode array 110 can be introduced into the patient in a first geometry (e.g. a collapsed geometry), and deployed and implanted in a second geometry (e.g. an expanded geometry).

ID 100 can comprise one, two, or more anchoring elements, anchor 150 shown, that can be configured to attach to and maintain the position of one or more components of ID 100 (e.g. maintain the position within a vessel). Anchor 150 can include one, two, or more hooks or barbs, adhesive (e.g. fibrin glue), and/or other anchoring elements. For example, anchor 150 can comprise one, two, or more scaffolding elements, such as one or more scaffolding elements comprising a stent-like structure which can be constructed and arranged to be positioned (e.g. and expanded) within a cardiac vessel and to maintain the position of ID 100 within the vessel. Each anchor 150 can comprise a structure (e.g. a stent-like structure) that is plastically deformable (e.g. configured to be expanded by an angioplasty balloon) and/or self-expanding (e.g. comprising self-expanding nickel titanium alloy or other self-expanding material). In some embodiments, ID 100 is constructed and arranged to be implanted into a vein and/or an artery, such as when implanted in the vein of Marshall. Anchor 150 can be constructed and arranged to maintain the position of ID 100 (e.g. at least a portion of ID 100) within a target blood vessel (e.g. the vein of Marshall) or other blood vessel (e.g. vein or artery) location. In some embodiments, one or more portions of ID 100 (e.g. a portion including one or more electrodes 111) is configured to be positioned in a blood vessel selected from the group consisting of: a coronary vein; a coronary artery; the vein of Marshall; the coronary sinus; the great cardiac vein; the anterior intraventricular vein; the middle cardiac vein; and combinations of these.

In some embodiments, a first portion of ID 100 (e.g. a portion comprising one or more electrodes 111) is configured to be implanted in a first cardiac vein or other blood vessel (e.g. as listed hereabove) and/a second portion of ID 100 (e.g. a portion comprising one or more electrodes 111 and/or controller 130) is configured to be implanted in a second cardiac vein or other blood vessel (e.g. as listed hereabove). In some embodiments, a first portion of ID 100 (e.g. a portion comprising one or more electrodes 111) is configured to be implanted in a first cardiac vein (e.g. as listed hereabove), and a second portion of ID 100 (e.g. a portion comprising one or more electrodes 111) is configured to be implanted at an epicardial location (e.g. as described herebelow). In some embodiments, ID 100 is configured to be implanted in a blood vessel and/or other anatomical location such that electrodes 111 deliver stimulation energy to the left atrium, left ventricle, and/or other cardiac tissue (e.g. to deliver stimulation energy to one, two, or more locations of the left atrium or other cardiac locations). In some embodiments, ID 100 is configured to be implanted in one, two, or more locations selected from the group consisting of: middle and/or medial epicardial locations on the posterior wall of left atrium; superomedial epicardial locations on the roof of the left atrium; middle epicardial locations on the anterior wall of left atrium; medial (proximal) and/or middle and/or lateral (distal) locations within the coronary sinus (e.g. when affixed adjacent to the left atrial epicardium for left atrial pacing, and/or affixed adjacent to the left ventricular epicardium for left ventricular pacing); epicardial locations at the apex of the left ventricle and/or right ventricle; medial and/or middle epicardial locations on the anterior wall of the left ventricle and/or right ventricle; lateral epicardial locations on the left-lateral wall of the left ventricle and/or the right-lateral wall of the right ventricle; medial epicardial locations over the interventricular septum between the left and right ventricles; medial and/or middle epicardial locations on the posterior wall of the left ventricle and/or right ventricle; and combinations of these. One or more parameters of ID 100 and/or anchor 150 can be selected (e.g. in manufacturing and/or from a kit of two or more devices 100) based on one or more properties of the vein of Marshall (e.g. the length and/or the diameter of the vein).

In some embodiments, electrodes 111 comprise a coating and/or a surface treatment (either or both, “coating” herein), such as a coating that is configured to enhance the recording ability of ID 100 via electrodes 111 and/or to enhance the pacing ability of ID 100. For example, electrode 111 can comprise one or more coatings that are configured to increase the surface area of electrodes 111, such as to enhance the recording ability of electrodes 111, such as to increase the charge-injection capacity for delivering a stimulation impulse, such as by lowering the resistive impedance and/or by increasing the capacitive impedance of electrodes 111.

ID 100 can comprise controller 130, which can be configured to perform various functions of ID 100. Controller 130 can comprise a microprocessor, memory, and/or other components that can be constructed and arranged to control, perform, and/or otherwise enable one or more functions of ID 100. In some embodiments, controller 130 comprises one or more algorithms, algorithm 135 shown. Controller 130 can be constructed and arranged to execute algorithm 135 and to thereby execute one or more functions of ID 100. In some embodiments, each electrode 111 of electrode array 110 is independently addressable (e.g. electrically connected to at least two wires, such as a ground wire and a power and/or data wire, the wires positioned between each electrode 111 and controller 130), such that signals (e.g. data and/or power) can be transmitted between controller 130 and each electrode 111, these signals delivered individually or collectively to one or more electrodes 111. Alternatively or additionally, controller 130 and/or electrode array 110 can be configured in a multiplexed arrangement, such that each electrode 111 can be individually addressed via a multiplexing component.

ID 100 can include transceiver 120. Transceiver 120 can be configured to communicate (e.g. wirelessly communicate) with one or more other components of system 10, for example, one or more additional implanted devices 100′, as well as EPD 200, CD 300, console 400, and/or or another component of system 10. Transceiver 120 can comprise a receiving and/or transmitting interface, antenna 125. Antenna 125 can comprise one, two, or more antennas of various shapes, for example, antenna 125 can comprise planar micro coils configured in various shapes.

ID 100 can include power module 140. Power module 140 can include one or more power-generating, power-harvesting, power-storing, and/or other power-supplying components configured to deliver energy to ID 100. Power module 140 can be configured to provide power to one or more components of ID 100. In some embodiments, power module 140 comprises one or more batteries, capacitors, and/or other power-storing devices. In some embodiments, ID 100 does not include a battery (i.e. a source of power that is generated by an electrochemical reaction), for example, when power module 140 is configured to harvest power (e.g. configured to harvest power transmitted wirelessly from EPD 200), and power module 140 is configured to directly provide the harvested power to power the various components of ID 100. Power module 140 can be constructed and arranged to “harvest” power from kinetic motion, for example, from kinetic motion of heart tissue when at least a portion of ID 100 is positioned on and/or within the heart. In some embodiments, power module 140 comprises one or more piezoelectric components configured to convert kinetic energy to electrical energy.

In some embodiments, implantable device 100 comprises patient sensor 160 shown. Patient sensor 160 can comprise one, two or more sensors selected from the group consisting of: accelerometer; position sensor; gravimetric sensor; pressure sensor; strain gauge; and combinations of these. System 10 can be configured to monitor one or more patient parameters based on information recorded by patient sensor 160, such as heartbeat, patient position, and/or patient activity.

ID 100 can include one or more functional elements, functional element 199 shown. Functional element 199 can comprise one, two, or more sensors selected from the group consisting of: pressure sensor such as blood pressure sensor; acoustic sensor; respiration sensor; gas sensor such as blood gas sensor; flow sensor such as blood flow sensor; temperature sensor; pH sensor; optical sensor; metabolic sensor; gravitational position sensor, gravitational orientation sensor, physical motion sensor; body position sensor; and combinations of these. In some embodiments, functional element 199 comprises one, two, or more transducers, such as an optical transducer (e.g. an LED). In some embodiments, functional element 199 comprises a fluid delivery assembly, such as an assembly configured to deliver a pharmaceutical drug or other agent to the patient. In these embodiments, functional assembly 199 can comprise a refill port, such as a port that can be accessed with a needle that is advanced through the patient’s skin into functional assembly 199 in order to refill functional assembly 199 with additional agent to be delivered to the patient. In some embodiments, functional assembly 199 is configured to deliver energy to the patient, such as thermal energy, electrical energy, magnetic energy, radioactive energy, sound energy (e.g. ultrasound energy), light energy, mechanical energy, and/or chemical energy that is delivered to the patient.

In some embodiments, ID 100 includes one or more circuit boards comprising one or more components of ID 100, for example electrode array 110, transceiver 120, controller 130, and/or other components of ID 100. In some embodiments, one or more circuit boards of ID 100 comprise flexible circuit boards, such as flexible printed circuit boards.

System 10 can be configured to both monitor one or more patient parameters and to treat the patient based on the monitored parameters. For example, system 10 can be configured to monitor (e.g. via electrode array 110) and analyze (e.g. via controller 130) electrograms recorded by ID 100, and to pace and/or otherwise stimulate tissue if atrial fibrillation (AF) is detected. In some embodiments, system 10 is configured to monitor and/or record one, two, or more of electrophysiological activity, patient temperature, heartbeat information, and/or another patient parameter.

Implantable device 100 can comprise one or more portions, such as a first portion implanted in a first location (e.g. a vein or other blood vessel), and a second portion implanted at another location (e.g. the epicardial surface of the heart or other anatomical location within the patient). Each portion can include one or more electrodes 111. In some embodiments, one portion (e.g. implanted in a coronary vein) comprises at least one, two or more electrodes 111, and another portion (e.g. positioned proximate but outside the heart) comprises at least controller 130.

External patient device 200 (EPD 200) can be constructed and arranged to be worn by the patient, such as when positioned on the skin of the patient (e.g. when EPD 200 is temporarily adhered or otherwise temporarily attached to the patient’s skin), and/or when inserted in and/or otherwise attached to the patient’s clothing. In some embodiments, EPD 200 includes attachment assembly 280. Attachment assembly 280 can include an adhesive, such as an adhesive patch, configured to adhere EPD 200 to the patient’s skin for at least 6 hours, such as at least 12 hours, or at least 24 hours (e.g. before the adhesive patch must be replaced). Alternatively or additionally, attachment assembly 280 can comprise a harness, clip, specialized garment, or other non-adhesive based tool for positioning EPD 200 proximate the patient (e.g. proximate the location where ID 100 is implanted in the patient). For example, attachment assembly 280 can comprise a chest strap constructed and arranged to hold EPD 200 over the patient’s heart, for example when ID 100 is implanted onto the epicardial surface of the patients left atrium.

EPD 200 can include transceiver 220. Transceiver 220 can be configured to communicate (e.g. wirelessly communicate) with one or more components of system 10, for example, one or more implanted devices 100, and/or one or more additional external patient devices 200′, as well as CD 300, console 400, and/or other components of system 10. Transceiver 220 can comprise a receiving and/or transmitting interface, antenna 225. EPD 200 can be constructed and arranged to transmit power and/or data to one or more implantable devices 100, such as by transmitting a radio frequency (RF) energy from antenna 225, through the skin of the patient, towards ID 100, and ID 100 can be constructed and arranged to harvest the RF energy and/or receive the RF data via antenna 125. In some embodiments, EPD 200 is constructed and arranged to receive data from one or more implantable devices 100, such as when transceiver 120 is constructed and arranged to transmit RF data to EPD 200.

EPD 200 can include one or more user interfaces, user interface 250 shown. User interface 250 can include one or more user input and/or user output components, for example, one or more: displays, indicators (e.g. LEDs), speakers, buttons, microphones, and/or other user interface components. In some embodiments, EPD 200 includes one or more functional elements, functional element 299 shown. Functional element 299 can include one or more sensors and/or transducers. In some embodiments, functional element 299 comprises a fluid delivery assembly, such as an assembly configured to deliver a pharmaceutical drug or other agent to the patient (e.g. through the skin of the patient). In these embodiments, functional assembly 299 can comprise a refill port, such as a port that can be accessed with a needle in order to refill functional assembly 299 with additional agent to be delivered to the patient. In some embodiments, functional assembly 299 is configured to deliver energy to the patient, such as thermal energy, electrical energy, magnetic energy, radioactive energy, sound energy (e.g. ultrasound energy), light energy, mechanical energy, and/or chemical energy that is delivered to the patient.

EPD 200 can include processing unit 210 which can be configured to perform one or more functions of EPD 200. Processing unit 210 can include one or more algorithms, algorithm 215 shown. In some embodiments, processing unit 210 analyzes data (e.g. via algorithm 215) received from ID 100. For example, EPD 200 can receive data from ID 100, process (e.g. mathematically process) the information received via algorithm 215 (e.g. to determine if pacing should be performed, and to determine the parameters of stimulation energy to be delivered), and send information and/or power to ID 100 based on the processed information.

CD 300 can include one or more catheters and/or or one or more surgical tools for delivering ID 100 into the patient. Additionally, CD 300 can include one or more devices configured to diagnose and/or treat the patient, such as to perform a diagnosis and/or a treatment during a clinical procedure in which ID 100 is implanted into the patient. For example, CD 300 can comprise a cardiac mapping catheter which can be used to collect data (e.g. data to be processed by console 400) such as to map the cardiac electrical activity of the heart. Additionally or alternatively, CD 300 can comprise an ablation catheter which can be used to ablate tissue (e.g. cardiac tissue). In some embodiments, system 10 can include one or more clinician devices 300 that are constructed and arranged to enable the clinician to perform: a mapping procedure, a tissue treatment procedure (e.g. an ablation procedure or other tissue treatment procedure), a multi-site pacing procedure, and/or an ID 100 implantation procedure (e.g. for continued, post procedural treatment of the patient).

In some embodiments, CD 300 comprises electrode array 310 shown, which can comprise one or more arrays of electrodes that can be inserted into the patient. CD 300 can include user interface 350 shown. User interface 350 can include one or more user input and/or user output components, for example, one or more: displays, indicators (e.g. LEDs), speakers, buttons, levers, microphones, and/or other user interface devices. In some embodiments, user interface 350 comprises a handle (e.g. a catheter handle) including one or more controls, such as a steering control.

In some embodiments, CD 300 includes transceiver 320. Transceiver 320 can comprise an assembly configured to communicate (e.g. wirelessly communicate) with one or more components of system 10, for example, one or more implanted devices 100, one or more external patient devices 200, console 400, and/or other components of system 10. Transceiver 320 can comprise a receiving and/or transmitting interface, antenna 325. In some embodiments, CD 300 includes one or more functional elements, functional element 399 shown. Functional element 399 can include one or more sensors and/or transducers. In some embodiments, functional element 399 comprises a fluid delivery assembly, such as an assembly configured to deliver a pharmaceutical drug or other agent to the patient. In these embodiments, functional assembly 399 can comprise a refill port, such as a port that can be accessed with a needle in order to refill functional assembly 399 with additional agent to be delivered to the patient. In some embodiments, functional assembly 399 is configured to deliver energy to the patient, such as thermal energy, electrical energy, magnetic energy, radioactive energy, sound energy (e.g. ultrasound energy), light energy, mechanical energy, and/or chemical energy that is delivered to the patient.

In some embodiments, system 10 includes a data storage and processing device, server 600. Server 600 can comprise an “off-site” server (e.g. outside of the operating room or other clinical site in which ID 100 is implanted), such as a server maintained by the manufacturer of system 10. Alternatively or additionally, server 600 can comprise a cloud-based server. Server 600 can include processing unit 610 shown, which can be configured to perform one or more functions of server 600. Processing unit 610 can include one or more algorithms, algorithm 615. Server 600 can be configured to receive and store various forms of data, such as: patient, procedural, device, and/or other information, data 620. Data 620 can comprise data collected from multiple patients (e.g. multiple patients treated with system 10), such as data collected during and/or after clinical procedures where device 100 was implanted into the patient. For example, data can be collected from ID 100, transmitted to EPD 200, and sent to server 600 for analysis. In some embodiments, one or more devices of system 10, such as EPD 200 and server 600, can communicate over a network, network 700 shown, for example, a wide area network such as the Internet. In some embodiments, network 700 includes a virtual private network (VPN) through which various devices of system 10 transfer data.

Algorithm 615 can be configured to analyze data 620. For example, algorithm 615 can be configured to analyze data 620 collected from multiple patients to identify similarities and/or differences in treatment parameters and patient results. In some embodiments, algorithm 615 comprises a machine learning and/or other artificial intelligence algorithm (“AI algorithm” herein) that can be configured to identify patterns in the correlations between treatment parameters and results based on data collected from multiple patients. In some embodiments, algorithm 615 analyzes patterns to determine better treatment parameters for one or more patients to be treated using system 10. For example, algorithm 615 can identify one or more patterns in the data (e.g. one or more patterns associated with efficacy of the treatment being delivered to the patient) by analyzing data 620 collected from many patients (e.g. tens of thousands of patients). Algorithm 615 can be further configured to use these patterns to determine whether a patient (e.g. in the set of patients from which the data was collected and/or in a new patient) is receiving sub-optimal treatment (e.g. the parameters associated with pacing and/or other energy being delivered could be modified to improve efficacy). System 10 (e.g. via algorithm 615) can be configured to alert the clinician of a patient receiving sub-optimal treatment, and to recommend (e.g. via CD 300, such as the clinician’s phone or computer) the parameters be adjusted. In some embodiments, the clinician may schedule an appointment to adjust the parameters (e.g. in person), or the parameters can be adjusted remotely, for example, when CD 300 is configured to adjust the parameters remotely via network 700. Alternatively or additionally, server 600 can adjust the parameter automatically (e.g. via network 700). In some embodiments, one or more parameters are automatically adjustable (e.g. within certain thresholds), while other parameters require clinician approval.

As described herein, system 10 can comprise one or more algorithms, such as algorithms 135, 215, 415, and 615 shown in FIG. 1. In some embodiments, one or more of these algorithms can comprise an AI algorithm. In some embodiments, one or more of these algorithms comprises a bias, such as a bias to tend toward classification of false positives and/or false negatives. In some embodiments, one or more of these algorithms comprises a bias toward false positive detection of an arrhythmia, such as to avoid not delivering stimulation energy when an arrhythmia is present (e.g. stimulate for all arrhythmia as well as some non-arrhythmia events that are classified as an arrhythmia event due to the false positive bias).

In some embodiments, device 100 comprises at least one sensing location (e.g. at least one electrode 111 is configured to sense or otherwise record the electrical activity of the heart). Additionally or alternatively, device 100 can comprise at least three pacing locations (e.g. at least two, or at least three electrodes 111 are configured to deliver energy to tissue to pace the heart). In some embodiments, controller 130 of device 100 includes a fibrillation and/or other arrhythmias detection algorithm (e.g. algorithm 135), that determines when a patient requires therapy (e.g. based on recorded electrical activity of the heart). Alternatively or additionally, algorithm 215 of processing unit of EPD 200 can comprise an arrhythmia detection algorithm.

In some embodiments, EPD 200 is configured to transmit a signal to ID 100 when pacing is required. For example, transceiver 220 can produce an electromagnetic and/or radiofrequency signal (e.g. a signal at a predetermined frequency) in response to the fibrillation/arrhythmia algorithm (e.g. algorithm 215) determining that pacing therapy is required. In some embodiments, ID 100 includes a stent-like structure (e.g. anchor 150) constructed and arranged to hold device 100 within a vessel (e.g. a cardiac vein) In some embodiments, device 100 is positioned within the vessel with at least one electrode 111 positioned on the vein wall, closest to the cardiac muscle.

In some embodiments, anchor 150 comprises a stent-like construction. Device 100 can comprise an elongate shape, with a proximal end and a distal end (e.g. where the distal end is placed distally of the proximal end when ID 100 is implanted into a vessel). In some embodiments, the overall shape of ID 100 is defined by the shape of anchor 150, which can comprise an elongate shape including a proximal end and a distal end. In some embodiments, the distal end of ID 100 is configured to be placed inside a vein where the diameter of the vein is slightly smaller than the diameter of the vein where the proximal end of ID 100 is placed. In some embodiments, anchor 150 comprises atapered design to accommodate this diameter delta. Today’s commercial stents have been used primarily to hold open an artery or vein, however in ID 100, anchor 150 is configured (e.g. solely configured) to anchor (e.g. chronically fixate) ID 100 inside the vessel in which it has been implanted.

In some embodiments, anchor 150 comprises a stent assembly constructed and arranged to be easily inserted into a body lumen. In some embodiments, anchor 150 comprises an expandable construction (e.g. a self-expanding construction), such as a construction that expands when advanced from the lumen of a delivery catheter.

In some embodiments, anchor 150 comprises a stent assembly constructed and arranged to provide a constant pressure on the walls of the lumen in its expanded state to ensure ID 100 is maintained in its implanted position.

In some embodiments, anchor 150 comprises a stent assembly comprising a flexible body constructed and arranged to hold ID 100 (e.g. at least one electrode 111 of ID 100) against the lumen wall located on the cardiac muscle side while maintaining the compliance of the lumen.

In some embodiments, anchor 150 comprise a stent assembly having a smooth adherent surface, such as a surface configured to prevent fatty deposits and oils from sticking to ID 100.

In some embodiments, anchor 150 comprises a stent assembly constructed and arranged to retain the position of ID 100 within the lumen without causing damage to the lumen.

In some embodiments, anchor 150 comprises a stent assembly having an atraumatic structure (e.g. a structure that is flexible and soft enough, and free from sharp edges, such as to allow placement in a vein or other blood vessel, without damaging the blood vessel.

In some embodiments, anchor 150 comprises a stent assembly for placement in a body lumen, constructed and arranged to retain ID 100 within the body lumen in an open position. Anchor 150 can include a cylindrical shell and a plurality of circular coils embedded within the cylindrical shell. In some embodiments, anchor 150 is changeable between a first unexpanded state for placement within the body lumen and a second expanded state for holding ID 100 within the body lumen once positioned therein. In some embodiments, the cylindrical shell is made of expandable materials.

As described herein, system 10 can be constructed and arranged to deliver multi-site stimulation. System 10 can be configured to deliver stimulation energy of specific waveshapes, amplitudes, polarities, and durations and at specific times and through one or more specific “channels” (e.g. electrical channels in which signals from electrodes 111 are received and processed), as determined by an algorithm of system 10, such as algorithm 135, 215, and/or 415, described herein. In some embodiments, system 10 delivers multi-site stimulation energy via ID 100 and/or CD 300. In some embodiments, system 10 performs an assessment of the patient’s tissue (e.g. the tissue proximate the multiple stimulation sites) and determines a set of patient-specific optimized pacing parameters based on the assessment (e.g. as performed by an algorithm of system 10). These parameters can be determined during a clinical procedure, and these parameters can be programmed into EPD 200 and/or ID 100 for configuring a future stimulation to be delivered (e.g. when EPD 200 and/or ID 100 are used chronically following the clinical procedure).

In some embodiments, implantable device 100 comprises an “appendage portion” (e.g. a sensing and/or energy delivery portion) that is configured to be implanted in the left atrial appendage (LAA) of the heart of the patient. In some embodiments, this appendage portion is configured to reduce the risk of embolization, such as to reduce the risk of clot formation and migration within and/or otherwise proximate the LAA, such as when the appendage portion is positioned within and/or otherwise proximate the LAA to fluidly isolate the LAA from the left atrium (LA). In some embodiments, system 10 is configured to record (e.g. continuously and/or intermittently record) one or more patient physiologic parameters, such as blood pressure within and/or at least proximate the heart from the appendage portion. For example, blood pressure of the left side of the heart can be monitored to diagnose and/or prognose one or more cardiac diseases. In some embodiments, system 10 is configured to record (e.g. continuously and/or intermittently record) one or more patient physiologic parameters, such as blood pressure within and/or at least proximate the heart from the appendage portion. For example, blood pressure of the left side of the heart can be monitored to diagnose and/or prognose one or more cardiac diseases (e.g. when functional element 199 comprises a pressure sensor). In some embodiments, system 10 is configured to sense and/or pace the heart (e.g. as described herein) from an appendage portion of ID 100 that is implanted in the patient’s left atrial appendage.

The present inventive concepts can relate to systems (including kits), devices, and methods for generating, charging and storing electrical energy. The systems and devices can include implantable wireless components, such as components configured to perform pacing (e.g. ventricular and/or atrial pacing), and/or defibrillating (e.g. ventricular and/or atrial defibrillation). In some embodiments, the present inventive concepts can include a generator component that provides programmable, semi-automatic and/or automatic charging. In some embodiments, the systems described herein provide a power generation function that is powered by an electromagnetic source positioned outside the body of the patient. This source can be configured to charge an implanted power source (e.g. a rechargeable battery and/or capacitor) that has been implanted in the patient. Energy can be produced (e.g. delivered) in various ways, for example via electromagnetic induction and/or via a piezoelectric effect. In some embodiments, a power supply is implanted that can be recharged via an external source of electromagnetic radiation, such as an optical, electrical, and/or magnetic source.

The present inventive concepts can be understood at several different levels. From the most generalized perspective, the embodiments of the present inventive concepts commonly share the protocol of first conducting atrial pacing from one or more pacing sites in the atrium, such as to maximize the extent of resynchronization of atrial tissue. The instantaneous atrial pacing rate delivered at the pacing site(s) can be based on current atrial fibrillation cycle length (AFCL) data, such as data sensed in real time or near-real time (“real time” herein) by the system. The various pacing regimens of the present inventive concepts can themselves terminate atrial fibrillation, or, at the minimum, these pacing regimens can significantly lower the energy requirements needed in an additional atrial defibrillation (ADF) shock-delivery tier of therapy. For example, after multi-site pacing has been conducted for a period of time, the left atrial tissue begins resynchronization, and monitoring the electrical activity of the left atrium can be used to determine when resynchronization has fully occurred (e.g. and when to stop).

Several pacing regimen options are encompassed at the incipient pacing tier of therapy in accordance with the current inventive concepts, while the particulars of the multi-site pacing tier of therapy are essentially the same regardless of which one of the inventive pacing regimens is being used in conjunction.

In some embodiments, a method for terminating atrial fibrillation includes a multi-site pacing regimen in which venously placed multi-site burst pacing is conducted in an asynchronous manner, whereby the pacing is delivered at each of the multiple pacing sites as an interval train of pulses delivered at a predetermined coupling interval set proportional to a common atrial fibrillation cycle length (AFCL) value. This multi-site pacing regimen brings larger regions of fibrillating atrial tissue into phase-lock via delivery of multi-site burst pacing level pulses over single site or continuous pacing. Once phase-lock is obtained via such asynchronous venously placed multi-site pacing, the venously placed multi-site burst pacing continues until the atrial chamber is reset to normal rhythm. The selection of the common AFCL used for setting the pacing rates of the multiple pacing sites can be set to be equal to the minimum (i.e. shortest in a temporal sense) local AFCL value determined among the sensed local atrial sites. The local AFCL values can be determined by counting the number of depolarization wavefronts to enter the given atrial site over a selected time period, and then calculating the median and/or mean AFCL value from that information. This approach is especially useful in instances in which different locally sensed AFCL values vary from one another.

Generally speaking, the greater the variance of electrophysiological properties between the different atrial locations to be paced (e.g. burst paced), the more sensitive the result achieved will be to the manner of choosing the best AFCL value for setting the asynchronized pacing rates. For instance, when the set of local AFCL values are grouped very closely together, the choice of the minimum AFCL as the basis for setting the pacing rate, in other words the time intervals between delivery of successive pacing pulses (also referred to herein as the “S1--S1 interval”) commensurately becomes less critical. Also, empirical studies have demonstrated that for a special case in which atrial sensing is performed from only a single site while multi-site asynchronously burst pacing the atrial tissue are used, that basing the pacing regimen on the AFCL sensed at the Bachmann’s Bundle alone is adequate to achieve the stated objectives of the present inventive concepts.

In some embodiments, using the venously placed multi-site pacing elements (e.g. elements including electrodes 111), synchronized pacing trains are delivered to various pacing sites (e.g. multiple pacing sites) in order to phase-lock tissue at a uniform S1--S1 interval, such as an interval that is proportionally set to 70-99%, such as 80-95%, of the minimum AFCL. Next, a premature “S2” pulse can be delivered, if still necessary after burst pacing, to terminate atrial fibrillation at a uniform time interval between the last burst pulse of the pulse train and the specific time thereafter when the premature pulse is delivered (also referred to herein as the “S1-S2 interval”), which can be proportionally set as 85-95% of the S1--S1 interval.

In some embodiments, a method for terminating atrial fibrillation includes a multi-site burst pacing regimen in which multi-site venous pacing is conducted in an asynchronous and/or synchronous manner, whereby the pacing is delivered concurrently and/or independently to different pacing sites of the atrium wall and/or to the heart from the vein of Marshal and/or coronary sinus. The pacing is delivered in an independently controlled manner to achieve an effect of advancing and broadening the region of tissue that is in the refractory state. The burst pacing is delivered at the multiple paced sites as an equal and/or unequal-interval (e.g. irregular interval) train of pulses. The pulses are delivered at a predetermined coupling interval set proportional to the locally determined atrial fibrillation cycle length (AFCL) value (e.g. as determined in real time). The burst pacing can also be delivered (e.g. independently delivered) at each of the multiple paced sites as a single pulse at a predetermined instant after a specific fiducial event has been detected from each of the local electrogram signals, respectively. These multi-site burst pacing regimens can also bring large regions of fibrillating atrial tissue into phase-lock via delivery of pacing level pulses alone. Once local phase-lock is obtained via such asynchronous and/or synchronous venously placed multi-site pacing electrodes, the timing of delivery of the pacing pulses can be adjusted to advance and broaden the region of tissue that is in the refractory state until activation is naturally suppressed throughout all paced regions of the atrium. The above process can be repeated if still necessary to terminate atrial fibrillation.

In some embodiments, using the asynchronous venously-located multi-site pacing regimen (e.g. from pacing energy delivered by multiple venously-placed electrodes 111), the local pacing burst pulses are delivered to the various pacing sites to phase-lock tissue at S1--S1 intervals set proportionally as 70-99%, preferably 80-95%, of the local AFCL. Next, a defibrillation shock can be delivered, if still necessary after pacing, to terminate atrial fibrillation at a uniform S1-S2 interval proportionally set as 85-95% of the minimum S1--S1 interval.

As described herein, multi-site burst pacing of the atrium can be conducted from an area outside the heart chamber, such as at a location that is adjacent to the atrial chamber, with a goal being to eventually entrain all atrial tissue by pacing alone. In these embodiments, the multi-site pacing devices can be placed in the Vein of Marshal, the Coronary Sinus, and/or other locations in the venous system of the heart.

In some embodiments, the present inventive concepts include a cardiac therapy system, such as an implantable micropacing chip assembly (e.g. including a stent) device, that is configured to implement pacing and ADF shock therapies described hereabove and otherwise herein.

In some embodiments, the present inventive concepts include a cardiac therapy system, such as a system including an implantable micropacing chip assembly, such as an assembly including a self-anchoring membrane device which can be capable of implementing the pacing and ADF shock therapies described hereabove and otherwise herein.

As compared to atrial defibrillation threshold (ADFT) without phase-lock being provided via pacing according to the present inventive concepts, the ADFT with phase-lock described herein significantly lowers, if not eliminates, pain or other discomfort of the patient.

| Ostium | Group I (n = 11) | Group II (n = 12) | Mean (n = 23) | P |
|---|---|---|---|---|
| Left Atrial Oblique Vein | 1.56 mm ± 0.42 mm | 1.0 mm ± 0.0 mm | 1.23 mm ± 0.38 mm | <0.01 |
| Coronary Sinus | 9.45 mm ± 1.97 mm | 7.08 mm ± 0.79 mm | 8.22 mm ± 1.88 mm | <0.05 |

Table 1 above lists mean diameters of measurements made from human patients of the left atrial oblique vein ostium (the vein of Marshall) and coronary sinus ostium as presented in EP Europace, Volume 9, Issue 10, October 2007, pages 915-919. “P” values are from an unpaired Studen’s t-test. Diameters of various components of implantable device 100 (e.g. substrate 101, anchor 150, and/or other components of device 100) can be sized to fit within, and/or engage the walls of one or more veins of the heart, as described herein.

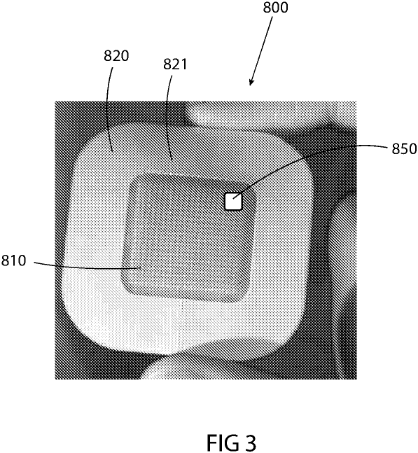

The various components of system 10 of FIG. 1 can be constructed and arranged as described herebelow in reference to FIGS. 3 and 6-15B.

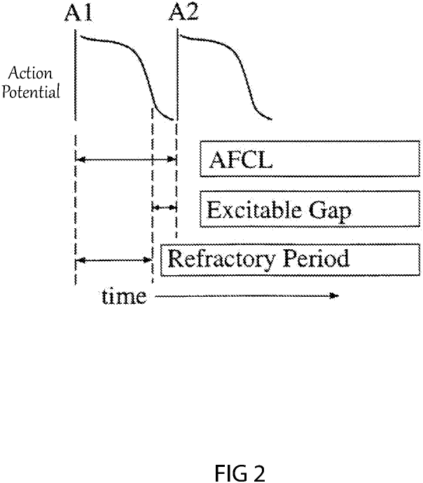

Referring to FIG. 2, a graphic of an action potential illustrating an atrial fibrillation cycle length is illustrated, consistent with the present inventive concepts.

Referring to FIG. 3, a photographic view of an array of electrodes, consistent with the present inventive concepts. Array 800 comprises an array of electrodes 810, which can be configured to record ECG signals and/or other electrical activity of patient tissue. Array 800 can comprise a border portion, border 820, which can comprise an adhesive 821 configured to attach array 800 to tissue of the patient. Electrodes 810 can comprise one or more needles, (e.g. microneedles) that can penetrate into the tissue onto which array 800 is placed. This penetration into tissue can yield enhanced ECG and/or other electrical signals to be recorded by electrodes 810. In some embodiments, array 800 is positioned on the patient’s skin, such as to provide skin-based ECG signals to system 10 (e.g. for a period of at least 1 day, or at least 3 days). In some embodiments, implantable device 100 comprises array 800 (e.g. electrode array 110 comprises array 800, such as when electrodes 111 comprise electrodes 810). In these embodiments, array 800 is implanted in the patient, such as when array 800 is positioned on an epicardial surface portion of the patient’s heart.

Array 800 can comprise a major axis of no more than 38 mm. Electrodes 810 can comprise at least 10 electrodes, such as at least 25, or at least 50 electrodes. Electrodes 810 can be configured to penetrate into a tissue surface with a penetration distance of at least 0.1 mm, no more than 3 mm, and/or approximately 0.5 mm. Electrodes 810 can comprise a conductive metal and/or polymer. Array 800 can comprise electronic module 850, such as a module configured to analyze the signals recorded by electrodes 810 and/or to transmit (e.g. via a wired or wireless transmission) data (e.g. recorded signal data and/or analyzed or otherwise processed recorded signal data) to another component of system 10. In some embodiments, electronic module 850 is configured to identify presence of AF, and transmit (e.g. wirelessly transmit) a detection signal to another component of system 10 such that energy can be delivered by ID 100 to treat the AF condition.

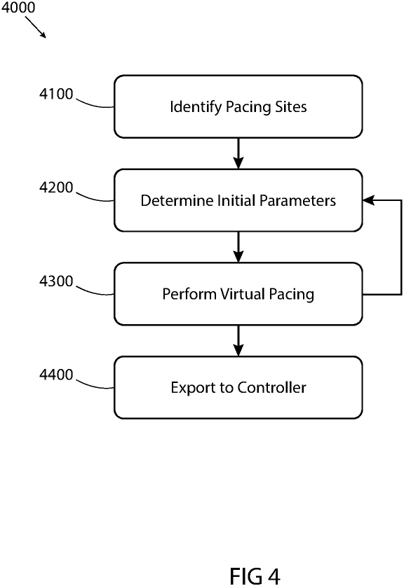

Referring to FIG. 4, a method for diagnosing and treating atrial fibrillation is illustrated, consistent with the present inventive concepts. Method 4000 of FIG. 4 comprises various steps for detecting and treating an arrhythmia, such as AF. Method 4000 is described using system 10 and its components of FIG. 1. In Step 4100 system 10 identifies multiple sites for delivering pacing energy, and/or parameters for delivering pacing energy (e.g. at the multiple sites). The pacing parameters can be based on the arrhythmias that arise in the atrial substrate of a patient following pacing evaluations and/or simulations performed by system 10. Pacing evaluation data can provide input to one or more algorithms (e.g. algorithm 415) that calculate pacing parameters based on the naturally deterministic and nonlinearly dynamical state of each of the paced regions of tissue throughout the atrium chamber. The evaluations and/or simulations can be based on multiple (e.g. 10) uniformly and/or non-uniformly distributed sites (e.g. sites along the vein of Marshall).

In Step 4200 an initial set of parameters is determined based on the data collected in Step 4100. This initial set of parameters comprises the number and target implant locations for electrodes (e.g. electrodes 111 of ID 100) to be used to treat the patient. The initial parameters of Step 4200 can also include energy delivery parameters (e.g. amplitude, phase, frequency, and the like) and/or timing of the energy delivery.

In Step 4300 virtual pacing of the target implant locations is performed, confirming that the atrial fibrillation is restored to normal sinus rhythm. The pacing protocol can return to Step 4200, where Steps 4200 and 4300 are repeated to assess whether a new arrhythmia will arise in the post-treatment substrate. These two steps can be repeated until system 10 determines that arrhythmias (e.g. AF) will no longer be sustained in the patient model (e.g. an atrial model), after which Step 4400 is performed.

In Step 4400, the successful results (e.g. stimulation protocol) of Step 4300 are provided to system 10 to treat the patient (e.g. via ID 100).

Referring to FIG. 5, a method for implanting a stent anchor is illustrated, consistent with the present inventive concepts. Method 5000 of FIG. 5 comprises various steps for implanting a treatment device into a patient. Method 5000 is described using system 10 and its components of FIG. 1. In Step 5100 system 10, the coronary sinus of a patient is accessed with a guide catheter (e.g. a CD 300 comprising a guide catheter). In Step 5200 a cardiac vein (e.g. the vein of Marshall) is accessed with a guidewire (e.g. a CD 300 further comprising at least one guidewire) that is advanced through the guide catheter. In Step 5300, electrodes (e.g. electrodes 111 of ID 100) are advanced over the guidewire into a vein of the patient’s heart (e.g. into the vein of Marshall). In Step 5400, one or more anchors (e.g. anchor 150) are introduced and/or deployed (e.g. if already present with electrodes 111) in a cardiac vein (e.g. the vein of Marshall). The deployed anchors 150 can comprise a deployed diameter (also referred to as “expanded diameter” herein) of at least 1.5 mm, and/or no more 4 mm, such as approximately 2 mm. The deployed anchors can be configured to position the one or more electrodes 111 against the wall of the vein in which electrodes 111 are placed. In Step 5500, one or more additional anchors (e.g. anchor 150) can be introduced and/or deployed (e.g. if already present with electrodes 111) in the coronary sinus. These deployed anchors can comprise a deployed diameter of at least 5 mm, such as a diameter between 8 mm and 10 mm. In some embodiments, deployed anchors 150 can comprise a deployed diameter of at least 10 mm, such as at least 15 mm (e.g. such as to induce stretch in the vein or other blood vessel in which the anchor is implanted).

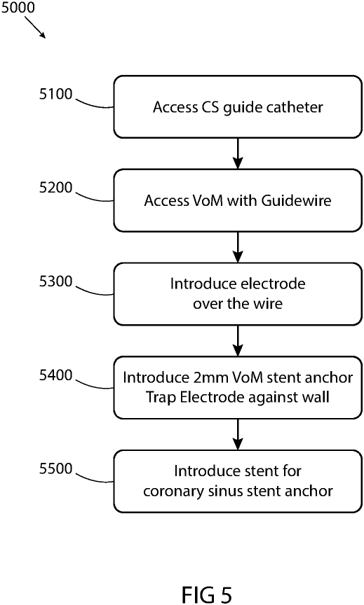

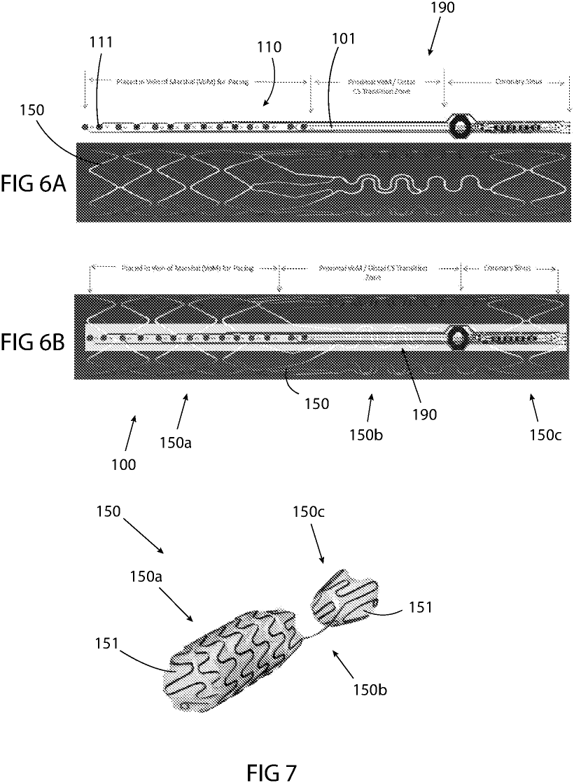

Referring to FIGS. 6A and 6B, side views of an assembly process for an implantable device are illustrated, consistent with the present inventive concepts. In FIG. 6A, two portions of an ID 100 are shown in an unassembled state. Implantable device 100 includes anchor 150 and assembly 190 as shown. Assembly 190 can comprise substrate 101 and electrode array 110 as shown, as well as other components described in reference to ID 100 of FIG. 1. In FIG. 6B, assembly 190 has been attached to anchor 150 as shown. Substrate 101 can comprise a flexible printed circuit board (PCB), such as a PCB onto which electrodes 111 and/or other electronic components can be operably attached and/or connected to each other. Anchor 150 of FIGS. 6A-B can comprise a first anchoring portion 150a configured to be positioned in a first blood vessel (e.g. the vein of Marshall), a second anchoring portion 150c configured to be positioned in a second blood vessel (e.g. the coronary sinus), and a connecting segment 150b therebetween. Anchoring portions 150a and 150c can comprise a stent-like geometry, as shown. Anchoring portions 150a and 150c can be configured to radially expand (e.g. in a self-expanding or balloon-expandable arrangement) to contact the walls of the blood vessel(s) in which they are implanted. Connecting segment 150b can comprise a circuitous geometry and/or other geometry that can axially extend and/or contract, such as to allow a range of different distances and angular orientations between the implant locations of portions 150a and 150c.

Referring to FIG. 7, a perspective view of an anchor is illustrated, consistent with the present inventive concepts. Anchor 150 of FIG. 7 can comprise a first anchoring portion 150a configured to be positioned in a first blood vessel (e.g. the vein of Marshall), a second anchoring portion 150c configured to be positioned in a second blood vessel (e.g. the coronary sinus), and a connecting segment 150b therebetween. Anchoring portions 150a and 150c can comprise a stent-like geometry, as shown. Anchoring portions 150a and 150c can be configured to radially expand (e.g. in a self-expanding or balloon-expandable arrangement) to contact the walls of the blood vessel(s) in which they are implanted. Connecting segment 150b can comprise a filament-like geometry (as shown) or other geometry that can flex to allow a range of different distances and angular orientations between the implant locations of portions 150a and 150c. Anchor 150 of FIG. 7 can comprise a covering, such as covering 151 shown. In some embodiments covering 151 comprises substrate 101 described in reference to FIG. 1 herein (e.g. a substrate comprising electrodes 111 and/or other components of an ID 100).

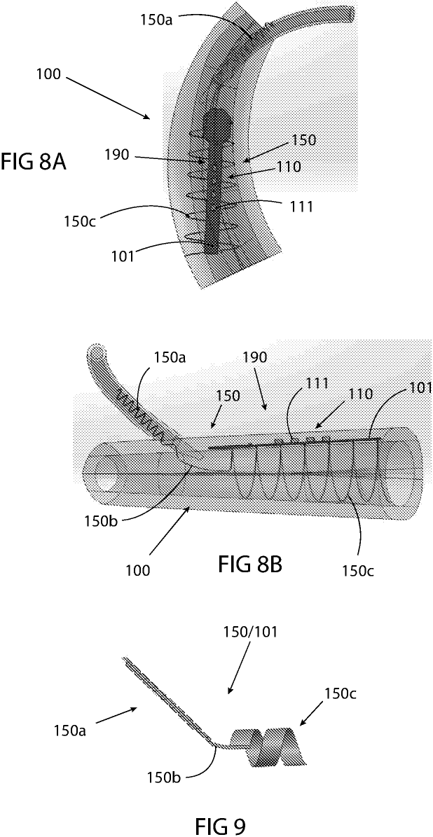

Referring to FIGS. 8A and 8B, top and side anatomical views of an implantable device are illustrated, respectively, consistent with the present inventive concepts. Implantable device 100 of FIGS. 8A-B includes anchor 150 and assembly 190 as shown. Assembly 190 can comprise substrate 101 and electrode array 110 as shown, as well as other components described in reference to implantable device 100 of FIG. 1. Assembly 190 can be attached to anchor 150 as shown. Substrate 101 can comprise a flexible printed circuit board (PCB), such as a PCB onto which electrodes 111 and/or other electronic components can be operably attached and/or connected to each other. Anchor 150 of FIGS. 8A-B can comprise a first anchoring portion 150a configured to be positioned in a first blood vessel (e.g. the vein of Marshall), a second anchoring portion 150c configured to be positioned in a second blood vessel (e.g. the coronary sinus), and a connecting segment 150b therebetween. Anchoring portions 150a and 150c can comprise a stent-like geometry, as shown. Anchoring portions 150a and 150c can be configured to radially expand (e.g. in a self-expanding or balloon-expandable arrangement) to contact the walls of the blood vessel(s) in which they are implanted. Connecting segment 150b can comprise a filament-like geometry (as shown) or other geometry that can flex to allow a range of different distances and angular orientations between the implant locations of portions 150a and 150c.

Anchor portions 150a and/or 150c can comprise a wire coil, as shown in FIGS. 8A and 8B. Alternatively, anchor portions 150a and/or 150c can comprise a ribbon coil as described herein in reference to FIG. 9. Anchor portion 150a of FIGS. 8A-B can comprise a diameter of approximately 1.5 mm, such as for implantation in the vein of Marshall. Anchor portion 150c of FIGS. 8A-B can comprise a diameter of approximately 9 mm, such as for implantation in the coronary sinus.

Referring now to FIG. 9, an anchor comprising a ribbon coil is illustrated, consistent with the present inventive concepts. Anchor 150 of FIG. 9 can comprise a first anchoring portion 150a configured to be positioned in a first blood vessel, a second anchoring portion 150c configured to be positioned in a second blood vessel, and a connecting segment 150b therebetween. Anchoring portions 150a and 150c can comprise a ribbon coil geometry, as shown. Anchor portion 150a can comprise a width of approximately 1.75 mm and anchor portion 150c can comprise a width of approximately 5 mm. Anchor portion 150a can comprise an expanded diameter of approximately 1.5 mm, such as for implantation in the vein of Marshall. Anchor portion 150c of FIG. 9 can comprise an expanded diameter of approximately 9 mm, such as for implantation in the coronary sinus. Anchoring portions 150a and 150c can be configured to radially expand (e.g. in a self-expanding or balloon-expandable arrangement) to contact the walls of the blood vessel(s) in which they are implanted. Connecting segment 150b can comprise a filament-like geometry (as shown) or other geometry that can flex to allow a range of different distances and angular orientations between the implant locations of portions 150a and 150c. Anchor 150 of FIG. 9 can comprise substrate 101 (e.g. anchor 150 and substrate 101 are the same component), such as when attached to substrate 101 are one or more electrodes 111 or other ID 100 components (not shown). In some embodiments, anchor 150 comprises substrate 101 as described in reference to FIG. 1, such as when substrate 101 comprises a flexible PCB comprising various electronic components operably attached to the PCB. Anchor portion 150a can comprise a length of approximately 2.5 cm (e.g. for placement in the vein of Marshall), and anchor portion 150c can comprise a length of approximately 19 mm (e.g. for placement in the coronary sinus).

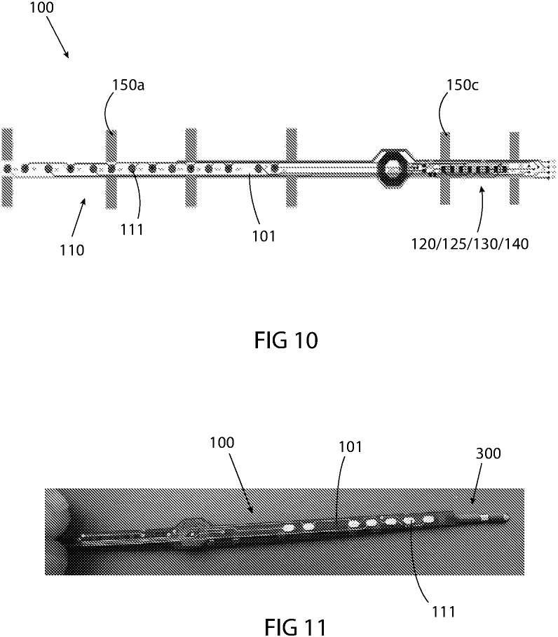

Referring now to FIG. 10, a top view of an implantable device is illustrated, consistent with the present inventive concepts. Implantable device 100 of FIG. 10 comprises anchor 150 comprising anchors 150a and 150c. Anchors 150a and 150c can each comprise projections (“wings”) that extend from substrate 101 as shown. The multiple projections of anchor 150a are positioned about electrode array 110 which includes electrodes 111. The multiple projections of anchor 150c are positioned about various electronic components of ID 100, such as transceiver 120, controller 130, and power module 140, each as described herein. Anchor portion 150a is configured for securing the associated portion of ID 100 in a first blood vessel (e.g. the vein of Marshall), and anchor segment 150c is configured for securing the associated portion of ID 100 in a second blood vessel (e.g. the coronary sinus). Anchor portions 150a and 150c can be constructed of flexible material, such as to allow portions 150a and 150c to curve and/or otherwise expand, and/or be resiliently biased in a curved (e.g. as shown in FIG. 14) and/or otherwise expanded geometry. Anchor portions 150a can be configured to cause electrodes 111 to make contact with the blood vessel wall into which electrodes 111 are implanted. Anchor portions 150a and/or 150c can be a continuous portion of substrate 101 (e.g. the same material as substrate 101 and/or an extension of the same component as substrate 101, such as when fabricated as a single component and/or a single assembly).

Implantable device 100 of FIG. 10 includes antenna 125 as shown, which is configured as an energy harvesting component of transceiver 120 as described herein. Antenna 125 can be configured to be positioned in a larger diameter segment of blood vessel (e.g. in the coronary sinus) than the segment in which electrodes 111 are placed (e.g. the vein of Marshall).

Referring to FIG. 11, a photographic view of an implantable device and a delivery catheter are illustrated, consistent with the present inventive concepts. Implantable device 100 can be inserted through a lumen of a delivery catheter, or alternatively, as shown in FIG. 11, ID 100 can be attached to the outer wall of a delivery catheter (e.g. CD 300 comprising a delivery catheter, such as a 2.7Fr delivery catheter). Implantable device 100 of FIG. 11 can be of similar construction and arrangement as ID 100 of FIG. 1 and otherwise herein. For example, ID 100 can comprise a device comprising a flexible PCB (e.g. substrate 101) including at least a portion that is configured to be positioned within and engage the walls of the vein of Marshall (e.g. a second portion configured to be positioned within and engage the wall of the coronary sinus). Device 100 can comprise multiple electrodes 111 that can be configured (e.g. programmed) individually and/or as groups of two or more electrodes to deliver energy (e.g. pacing energy) and/or record electrical signals.

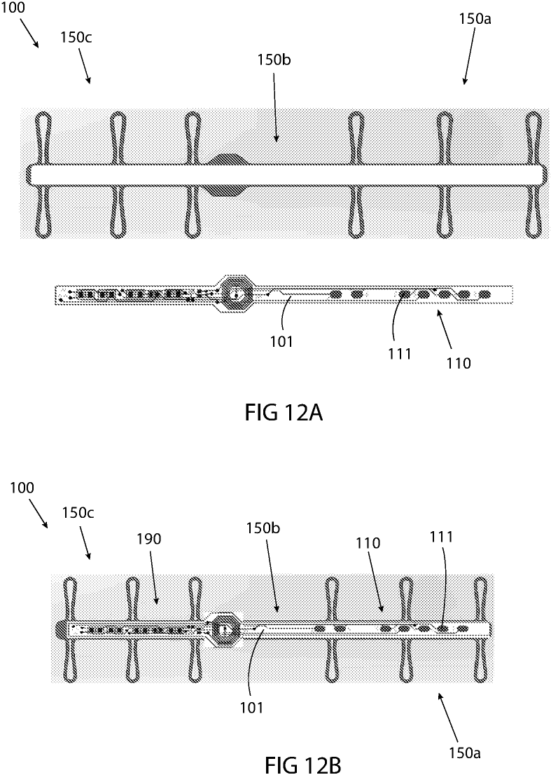

Referring to FIGS. 12A and 12B, side views of an assembly process for an implantable device are illustrated, consistent with the present inventive concepts. In FIG. 12A, two portions of an implantable device 100 are shown in an unassembled state. Implantable device 100 includes anchor 150 and assembly 190 as shown. Assembly 190 can comprise substrate 101 and electrode array 110 as shown, as well as other components described in reference to ID 100 of FIG. 1 and otherwise herein. In FIG. 12B, assembly 190 has been attached to anchor 150 as shown. Substrate 101 can comprise a flexible printed circuit board (PCB), such as a PCB onto which electrodes 111 and/or other electronic components can be operably attached and/or connected to each other. Anchor 150 of FIGS. 12A-B can comprise a first anchoring portion 150a configured to be positioned in a first blood vessel (e.g. the vein of Marshall), a second anchoring portion 150c configured to be positioned in a second blood vessel (e.g. the coronary sinus). Anchoring portions 150a and 150c can comprise projecting loops, as shown (e.g. loop geometry extensions of substrate 101). Anchoring portions 150a and 150c can be configured to radially expand (e.g. in a self-expanding or balloon-expandable arrangement) to contact the walls of the blood vessel(s) in which they are implanted.

In some embodiments, anchor portions 150a and/or 150c comprise nickel titanium alloy or a similar elastic material. In these embodiments, anchor portions 150a and/or 150c can be part of a frame surrounding at least a portion of substrate 101. In some embodiments, ID 100 comprises a laminate construction, and/or an overmold.

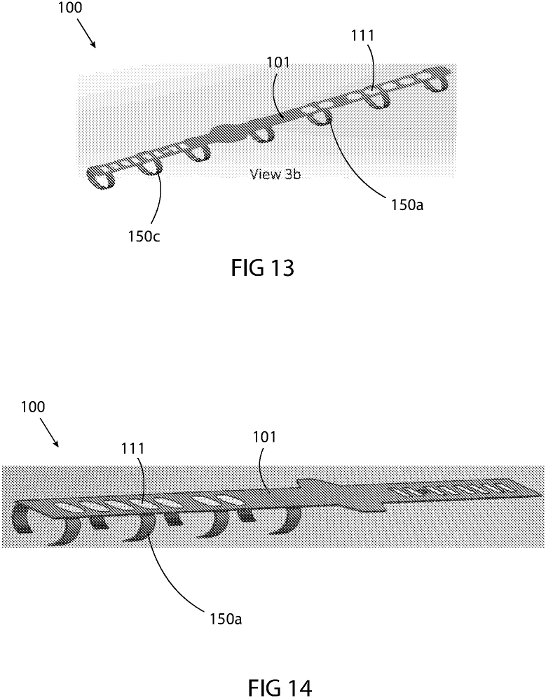

Referring now to FIG. 13, a perspective view of an implantable device including ring-shaped anchors is illustrated, consistent with the present inventive concepts. Implantable device 100 of FIG. 13 can be of similar construction and arrangement as ID 100 described in reference to FIG. 1 and otherwise herein. ID 100 comprises substrate 101 onto which electrodes 111 are positioned. Device 100 comprises anchor 150 comprising anchor portions 150a and 150c. Anchor portions 150a and/or 150c can comprise a ring-like construction as shown. In some embodiments, anchor portions 150a and/or 150c are ring-like projections extending from opposite sides of substrate 101 (e.g. the same, continuous, material as substrate 101).

Referring now to FIG. 14, a perspective view of an implantable device including curved anchors is illustrated, consistent with the present inventive concepts. Implantable device 100 of FIG. 14 can be of similar construction and arrangement as ID 100 described in reference to FIG. 1 and otherwise herein. ID 100 comprises substrate 101 onto which electrodes 111 are positioned. Device 100 comprises anchor 150 comprising anchor portions 150a. Anchor portions 150a are configured for placement in a first blood vessel (e.g. the vein of Marshall), such as to position electrodes 111 against the wall of the blood vessel. Anchor 150 can further comprise anchor portions 150c, not shown but positioned on the opposite end of ID 100 and configured for placement in a second blood vessel (e.g. the coronary sinus). In some embodiments, anchor portions 150a and/or 150c are curved projections extending from opposite sides of substrate 101 (e.g. the same, continuous material of substrate 101).

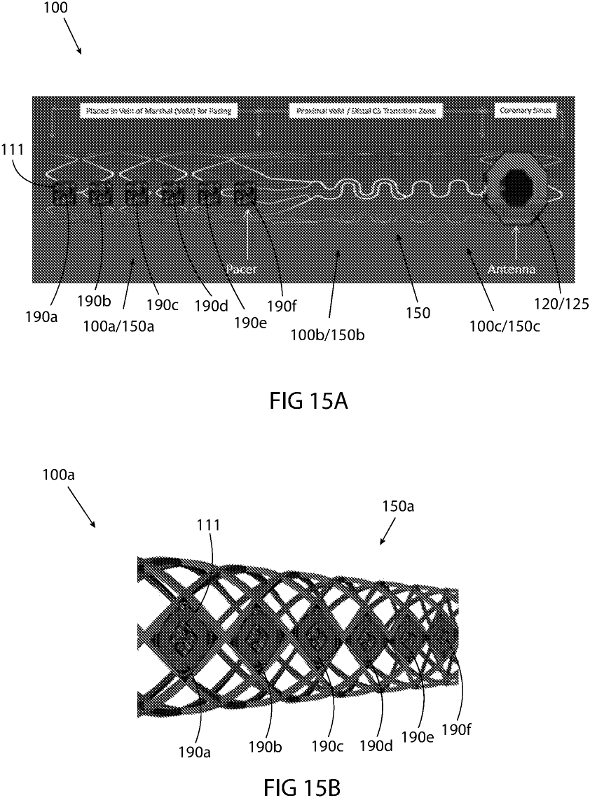

Referring now to FIGS. 15A and 15B, a top view of an implantable device, and a perspective view of a portion of the implantable device, respectively, are illustrated, consistent with the present inventive concepts. Implantable device 100 of FIG. 15 can be of similar construction and arrangement as ID 100 described in reference to FIG. 1 and otherwise herein. ID 100 comprises a first portion 100a including anchor portion 150a, a second portion 100c including anchor portion 150c, and a middle portion 100b comprising connecting segment 150b. FIG. 15B just shows first portion 100a. Device 100 comprises an assembly 190 comprising multiple assemblies, such as assemblies 190a, 190b, 190c, 190d, 190e, and 190f as shown. Assemblies 190a-f each comprise a stand-alone assembly (a “micropacing chip” as described herein) including at least one or more electrodes 111, and associated control circuitry to independently control those one or more electrodes 111 (e.g. to record electrical activity and/or to deliver pacing and/or other stimulating energy). ID 100 further comprises transceiver 120 including antenna 125 as shown. In some embodiments, one or more assemblies of assemblies 190a-f comprise an antenna 125 (e.g. and associated transceiver 120 components). In these embodiments, energy harvested by the antennas 125 of one or more of the assemblies 190a-f can be utilized and/or stored in a cumulative fashion (e.g. added together).

1. A system for treating a cardiac condition of a patient, the system comprising:
an implantable device configured to deliver energy to the patient’s heart;
an external patient device configured to wirelessly communicate with the implantable device; and
a clinician device configured to implant the implantable device in the patient,
wherein the cardiac condition treated by the system comprises atrial fibrillation.

2-76. (canceled)
